(19) United States
(12) Patent Application Publication
(10) Pub. No.: US 2016/0235808 A1
(43) Pub. Date: Aug. 18, 2016

(54) Treatment of Hepatitis C Virus Infection with Alisporivir

(71) Applicants: Claudio Avila, Basel (CH); Rafael Crabbe, Bursins (CH)

(72) Inventors: Claudio Avila, Basel (CH); Rafael Crabbe, Bursins (CH)

(21) Appl. No.: 15/140,607

(22) Filed: Apr. 28, 2016

Related U.S. Application Data
(63) Continuation of application No. 14/008,681, filed on Sep. 30, 2013, now abandoned.
(60) Provisional application No. 61/474,946, filed on Apr. 13, 2011.

(51) Int. Cl. A61K 38/13; A61K 31/7056; A61K 38/21

(52) U.S. Cl. CPC A61K 38/13; A61K 38/212; A61K 31/7056

(57) ABSTRACT

The invention concerns the use of cyclophilin inhibitors in the treatment of Hepatitis C virus infection.

The present disclosure relates to a non-immunosuppressive cyclosporin which binds to cyclophilin, which are cyclophilin inhibitors, in particular to their pharmaceutical use of in the treatment of Hepatitis C virus infection.

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A (CsA).

Cyclosporins which bind strongly to cyclophilin but are not immunosuppressive have been identified. PCT/EP 2004/009804, WO 2005/021028, or WO 2006/071619 disclose non-immunosuppressive cyclosporins which bind to cyclophilin have also been found to have an inhibitory effect on Hepatitis C virus (HCV). WO 2006/038088, incorporated herein by reference in its entirety, describes methods and compositions for the use of alisporivir in the treatment of HCV. Alisporivir (DEB025 or Debio-025) is a cyclophilin (Cyp) inhibitor and its mode of action as an anti-HCV agent is via inhibition of host proteins, in particular of cyclophilin A, that are directly involved in HCV replication.

Hepatitis C virus (HCV) is an enveloped single stranded (+) RNA virus that belongs to the separate genus Hepacivirus of the family Flaviviridae. HCV causes acute and chronic liver disease, including chronic hepatitis, cirrhosis, and hepatocellular carcinoma. Worldwide more than 170 million people are chronically infected with HCV and are thus at increased risk of developing serious life-threatening liver disease.

The current standard of care in HCV patients consists of a combination of interferon and ribavirin. Treatment duration and ribavirin dose depend on the genotype treated. Sustained viral response (SVR) in patients with genotypes 2 and 3 after standard of care treatment reaches 80-90%, but only 40-50% in patients with genotype 1. Furthermore, side effects are significant and include myalgia, arthralgia, headache, fever, severe depression, leucopenia and haemolytic anaemia.

As a result, there is currently a large proportion of chronic HCV infected patients that have failed previous treatment and that are in high need for new treatment modalities that would allow them to achieve SVR and halt the further evolution of their chronic liver disease. Persistent infection by HCV, which has been identified as the major causative agent of non-A, non-B hepatitis has been considered closely related to liver diseases such as chronic hepatitis, liver cirrhosis or hepatocellular carcinoma. The development of these liver diseases is a major public health problem.

Despite the positive indications in the art of the use of CsA and non-immunosuppressive cyclosporins in treatment of HCV, there is a significant class of HCV patients that remains refractory to the current standard of care therapies. Non-responders to standard of care treatment represent an important medical challenge. For these patients, no alternative antiviral regimen is available and a substantial percentage of them will develop progressive disease with cirrhosis and end stage liver disease, sometimes complicated by hepatocellular carcinoma resulting in orthotopic liver transplantation. Thus, despite existing therapies, there remains a significant need for methods and compositions for the treatment of HCV.

Failure to achieve a can also be a consequence of relapse. Relapse is defined as reappearance of HCV RNA during the post-treatment follow-up after having achieved undetectable HCV RNA at end of treatment. Relapse is a significant clinical problem, especially for the genotype 1 chronic hepatitis C population. Therefore, there is a need to provide therapy regimens which will improve efficacy for both, relapsers and non-responders patients.

Surprisingly we have found out that cyclophilin inhibitors, in particular alisporivir, can be used effectively in the treatment of HCV. In particular, we have found that satisfactory treatment results of Hepatitis C virus genotype 1 infection in relapser or non-responder patients to standard of care can be obtained when using alisporivir.

Accordingly, the present invention provides new anti-HCV treatments using alisporivir, in particular methods of treating hepatitis C virus genotype 1 infection in a relapser or non-responder patient comprising administering to the patient alisporivir, during an initial phase in an amount of about 600 mg twice a day; followed by administering alisporivir during a second phase in an amount of about 600 to about 1000 mg once per day.

The invention further provides alisporivir for use in the treatment or prevention of Hepatitis C virus genotype 1 infections or HCV induced disorders in a relapser or a non-responder patient.

### SUMMARY OF THE DISCLOSURE

Further, the following is described:

1.1 A method for preventing or treating Hepatitis C infections or HCV induced disorders in a relapser or a non-responder patient, comprising administering to said patient alisporivir during an initial phase in an amount of about 600 mg twice a day; followed by administering alisporivir during a second phase in an amount of about 600 to about 800 mg once per day.

1.2 A method for inhibiting HCV replication in a relapser or a non-responder, comprising administering alisporivir during an initial phase in an amount of 600 mg twice a day; followed by administering alisporivir during a second phase in an amount of about 600 to about 800 mg once per day.

1.3. A method for preventing or treating Hepatitis C infections or HCV induced disorders in a relapser or a non-responder patient, comprising administering to said patient alisporivir in an amount of about 400 mg twice a day.

1.4. Any method as defined above, wherein the HCV infection is Hepatitis C virus genotype 1 infection.

2. Use of alisporivir in the preparation of a pharmaceutical composition for use in any method as defined above.

3. Use of alisporivir in the preparation of a medicament for use in any method as defined above.

4. A pharmaceutical composition for use in any method as defined above, comprising alisporivir, together with one or more pharmaceutically acceptable diluents or carriers therefor.

5. A therapeutic regimen comprising administering alisporivir during an initial phase in an amount of about 600 mg, twice per day followed by administering alisporivir during a second phase in an amount of about 600 to about 800 mg per day and wherein alisporivir is administered in combination with standard of care throughout the initial and second phases.

6. A package comprising the pharmaceutical composition comprising alisporivir as defined above, in combination with instructions to administer said composition during an initial phase in an amount of about 600 mg twice a day; followed by administering alisporivir during a second phase in an amount of about 600 to about 800 mg per day.

7. A kit for the treatment of chronic hepatitis C infection.

Also contemplated herein is a method of reducing the HCV RNA in a relapser or a non-responder patient comprising administering to the patient: alisporivir, an interferon; and ribavirin in which alisporivir is administered during an initial phase in an amount of about 600 mg twice a day; followed by administering alisporivir during a second phase in an amount of about 600 or about 800 mg once per day.

Additional embodiments of the present invention relate to methods of treating hepatitis C genotype 1 infections in a patient that is resistant to standard of care therapy for HCV treatment comprising administering to the patient: alisporivir in combination with standard of care, wherein alisporivir is administered during an initial phase in an amount of about 600 mg twice a day; followed by administering alisporivir during a second phase in an amount of about 600 to about 800 mg once per day.

Also contemplated herein is a pharmaceutical combination comprising a first pharmaceutically acceptable formulation comprising alisporivir, a second pharmaceutically acceptable formulation comprising an interferon and a third pharmaceutically acceptable formulation comprising ribavirin, wherein the first, second and third formulations are packaged in a kit for the treatment of chronic hepatitis C infection.

### DETAILED DESCRIPTION OF THE DISCLOSURE

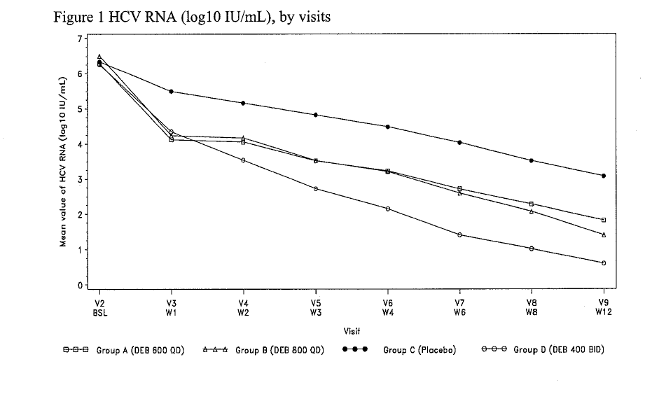

FIG. 1 shows the HCV RNA (log 10 IU/mL), by visits for all treatment arms, up 12 weeks of treatment.

In the above embodiments and throughout this specification, the standard of care treatment is a treatment that is used to treat Hepatitis C infections. The currently used standard of care treatment involves administration of interferon, in particular pegylated interferon in combination with ribavirin.

In the above embodiments and throughout this specification, the initial phase is a period of 3, 4, 5, 6, or 7 days. Preferably the initial phase is a period of at least 3 days, preferred of 7 days.

In the above embodiments and throughout this specification, the second phase is a period of 23, 47 or 71 weeks. Preferably the second phase is a period of 47 weeks.

In the present application, the term “relapser” is intended to mean a patient or subject who relapse to standard of care treatment for HCV. More specifically, a relapser to standard of care patient is a patient with undetectable HCV RNA<10 IU/mL levels at the end of treatment which become detectable again during post-treatment follow up, at any time point after treatment end, in particular within 24 weeks of post-treatment follow-up after previous undetectable HCV RNA at end of treatment.

In the present application, the term “non-responder” is intended to mean a patient or subject who is a non-responder to standard of care treatment for HCV. More specifically, a non-responder to standard of care patient is a patient who has not responded to treatment with standard of care given over a 12 weeks treatment period. The non-responder to standard of care includes the following subsets of patients—null responders and partial responders.

Typically, a patient who has a “null response” may, for example, be defined as one in whom the HCV-RNA reduction is observed to be less than 2 log 10 IU/mL after 12 weeks of treatment with standard of care.

A patient that has a “partial” response or partial responder is one in whom the HCV-RNA reduction of more than 2 log 10 IU/mL is observed after 12 weeks of treatment with standard of care but the HCV-RNA is still detectable at the end of treatment.

In the present invention, an interferon may be pegylated or non-pegylated and may include interferons such as: Intron-A®, interferon alfa-2b (Schering Corporation, Kenilworth, N.J.); PEG-lntron®, peginteferon alfa-2b (Schering Corporation, Kenilworth, N.J.); Roferon®, recombinant interferon alfa-2a (Hoffmann-La Roche, Nutley, N.J.); Pegasys®, peginterferon alfa-2a (Hoffmann-La Roche, Nutley, N.J.); Berefor®, interferon alfa 2 available (Boehringer lngelheim Pharmaceutical, Inc., Ridgefield, Conn.); Sumiferon®, a purified blend of natural alpha interferons (Sumitomo, Japan); Wellferon®, lymphoblastoid interferon alpha n1 (GlaxoSmithKline); Infergen®, consensus alpha interferon (InterMune Pharmaceuticals, Inc., Brisbane, Calif. and Amgen, Inc., Newbury Park, Calif.); Alferon®, a mixture of natural alpha interferons (Interferon Sciences, and Purdue Frederick Co., Conn.); Viraferon®; and combinations of these interferons.

Conjugated interferons that may be used include, for example, Albuferon (Human Genome Science) which is conjugated to human albumin. Interferon conjugated to a water-soluble polymer or polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Interferon-polymer conjugates are described in U.S. Pat. No. 4,766,106, U.S. Pat. No.4,917,888, EPA 0 236 987, EPA 0 510 356 and WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Other forms of interferons include interferon beta, gamma, tau and omega, such as Rebif (Interferon beta 1a) by Serono, Omniferon (natural interferon) by Viragen, or Omega Interferon by Boehringer Ingelheim. Oral interferons such as oral interferon alpha by Amarillo Biosciences.

Additional examples of interferons that may be used include pegylated interferon alpha, for example pegylated interferon α-2a, pegylated interferon α-2b, pegylated consensus interferon or pegylated purified interferon-a product. Pegylated interferon α-2a is described in European Patent 593,868 (incorporated herein by reference in its entirety) and commercially available e. g. under the trade name PEGASYS® (Hoffmann-La Roche). Pegylated interferon-α-2b is described, e.g. in European Patent 975,369 (incorporated herein by reference in its entirety) and commercially available e.g. under the trade name PEG-INTRON A ® (Schering Plough). Pegylated consensus interferon is described in WO 96/11953 (incorporated herein by reference in its entirety).

In preferred embodiments, the interferon used in the methods of the invention is pegylated interferon. In other embodiments, the interferon is selected from the group consisting of interferon alpha-2a, Interferon alpha-2b, a consensus interferon, a purified interferon alpha product or a pegylated interferon alpha-2a, pegylated interferon alpha-2b, and pegylated consensus interferon, a mixture of natural alpha and combinations thereof.

Preferably the methods using interferon alpha use a pegylated interferon alpha-2b and the amount of pegylated interferon alpha-2b is from 0.5 to 2.0 micrograms/kilogram per week on a weekly, three times a week, every other day or daily basis.

As used herein, “microgram/kilogram” means microgram drug per kilogram body weight of the mammal—including man—to be treated.

As used herein, the term “treatment” or “treat” refer to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during HCV therapey. A therapeutic regimen may include an induction regimen and a maintenance regimen.

The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “about”, unless the context dictates otherwise, is used to mean a range of + or −10%.

In other embodiments, the interferon alpha is a pegylated interferon alpha-2a and the amount of pegylated interferon alpha-2a administered is from 20 to 250 micrograms/kilogram per week on a weekly, three times a week, every other day or daily basis. Preferably, the interferon peg-IFNa2a is administered at an amount of 180 micrograms once per week.

In specific embodiments, the exemplary interferon used in the methods herein is interferon selected from the group consisting of Intron-A®; PEG-lntron®; Roferon®; Pegasys®; Berefor®; Sumiferon®; Wellferon®; Infergen®; Alferon®; Viraferon®; Albuferon® (Human Genome Science); Rebif; Omniferon; Omega and combinations thereof.

In some embodiments, the patient may be administered ribavirin or a ribavirin derivative (e.g., a ribavirin analog or prodrug, such as ribamidine, taribavirin (viramidine), ICN 17261, molecules disclosed in WO/2008/052722, which is incorporated by reference in its entirety, etc.).

In some embodiments, ribavirin is administered at between about 800 mg to about 1200 mg per day, e.g., 800 mg, 900 mg, 1000 mg, 1100 mg, 1200 mg per day. In some embodiments, ribavirin is administered based on the weight of the patient.

In another embodiment, alisporivir may be administered with additional agents of the standard of care that promote the antiviral efficacy of the therapy treatment. The standard of care may include additional agents that promote the antiviral efficacy of the therapy treatment, such as substrate-based protease inhibitors of HCV NS3-4A serine protease, non-substrate-based NS3 protease inhibitors; phenanthrenequinones, thiazolidines and benzanilides, nucleosides analogs, antisense molecules directed against HCV genome or any cellular component that is required for viral replication, vaccine or antibody-based approaches to HCV treatment. Direct acting antiviral agents, is used herein to mean agents that interfere with specific steps in the hepatitis C virus (HCV) replication cycle. Such agents may be, e.g., ribavirin derivatives, protease inhibitors, polymerase inhibitors (e.g., nucleoside and non-nucleoside inhibitors), and cyclophillin inhibitors. Exemplary antiviral include: boceprevir, telaprevir, ABT-072, ABT-450, ABT-333 by Abbott, ACH1625 by Achillion, ANA598 by Anadys Pharmaceuticals, AZD-7295 by AstraZeneca, BI201335, BI207127 by Boehringer Ingelheim Pharma, BMS650032, BMS790052, BMS791325, BMS824383 by Bristol Myers Squibb, Clemizole by Eiger BioPharmacetucials, Filibuvir by Pfizer, GS9190 (Tegobuvir), GS9256 by Gilead, IDX375 by Idenix, INX-189 by Inhibitex, PSI-7851, PSI-938 by Pharmasset, PSI-7977, RG7128 by Pharmasset/Genethec, PPI-461 by Presidio RG7227 (Danoprevir) by InterMune/Genentech, SCH900518 (Narlaprevir), Vaniprevir by Merck, TMC435 by Medivir/Tibotec, VX-222, VX-759, VX-500, VX-916 by Vertex.

In some embodiments, alisporivir may be administered once per day (daily), twice per day, three times per day, every other day, every three days, weekly (once per week), once every other week, once every three weeks, once monthly, etc.

In one embodiment, the present invention further provides alisporivir for use in combination with standard of care in treatment of a Hepatitis C virus infected patient, the alisporivir to be administered in an amount of about 400 to about 600 mg (e.g., about 350 mg, about 400 mg, about 450 mg, about 500 mg, about 550 mg, about 600 mg, about 650 mg) twice per day.

As used herein “twice per day” means twice in any period of about 24 hour period.

As used herein “once per week” is used to mean once in any period of about seven days.

In still another aspect, alisporivir is to be administered for up to 24, 48 or 72 weeks. As used herein “up to 24, 48 or 72 weeks” is used to mean that alisporivir is administered on a continuous basis (e.g., twice per day, once per week, etc.) for about 24 weeks, about 48 weeks, or about 72 weeks. It will be understood that therapy need not end at exactly the 24, 48 or 72 week time period. For example, therapy may end a day or a few days before the 24 week period, and still be an equivalent within the scope and spirit of the current disclosure.

In one embodiment, the present invention further provides alisporivir for use in combination with standard of care in treatment of a Hepatitis C virus genotype 1 infected relapser or a non-responder patient, the alisporivir being administered during an initial phase in an amount of about 600 mg, twice per day; followed by administering alisporivir during a second phase in an amount of about 600 to about 800 mg once per day. In still another aspect, the initial phase is a period of 7 days; the second phase is a period of 23, 47 or 71 weeks.

In one embodiment, the present invention further provides alisporivir for use in combination with an interferon (e.g., pegylated interferon alpha 2a or pegylated interferon alpha 2b) and ribavirin in treatment of a Hepatitis C virus genotype 1 infected relapser or non-responder patient, the alisporivir being administered during an initial phase in an amount of about 600 mg, twice per day for 7 days; followed by administering alisporivir during a second phase in an amount of about 600 or about 800 mg once per day for up to 23, 47 or 71 weeks.

In one embodiment, the present invention further provides alisporivir for use in combination with interferon and ribavirin in treatment of a Hepatits C virus genotype 1 infected relapser or non-responder patient, the alisporivir being administered during an initial phase in an amount of about 600 mg, twice per day for 7 days; followed by administering alisporivir during a second phase in an amount of 600 mg once per day for up to 71 weeks, preferably up to 23 weeks, most preferred up to 47 weeks.

In one embodiment, the present invention further provides alisporivir for use in combination with interferon and ribavirin in treatment of a Hepatitis C virus genotype 1 infected relapser or non-responder patient, the alisporivir being administered during an initial phase in an amount of about 600 mg, twice per day for 7 days; followed by administering alisporivir during a second phase in an amount of 800 mg once per day for up to 47 weeks.

In one embodiment, the present invention further provides alisporivir for use in combination with standard of care, preferably with pegylated interferon alpha-2a and ribavirin in treatment of a Hepatitis C virus genotype 1 infected relapser or non-responder patient, the alisporivir being administered in an amount of about 400 mg, for up to 24, 48 or 72 weeks.

In one embodiment, the present invention further provides alisporivir for use in combination with standard of care, preferably with pegylated interferon alpha-2a and ribavirin in treatment of a Hepatitis C virus genotype 1 infected relapser or non-responder patient, the alisporivir being is administered during an initial phase in an amount of about 600 mg, twice per day; followed by administering alisporivir during a second phase in an amount of about 600 to about 800 mg once per day for up to 23 or 47 or 71 weeks. In still another aspect, the pegylated interferon alpha-2a and is administered in an amount of 180 micrograms once per week.

In one embodiment, the present invention further provides alisporivir for use in combination with pegylated interferon alpha-2a and ribavirin in treatment of a Hepatitis C virus genotype 1 infected relapser or non-responder patient, the alisporivir being administered during an initial phase in an amount of about 600 mg, twice per day; followed by administering alisporivir during a second phase in an amount of about 600 to about 800 mg once per day for up to 23, 47 or 71 weeks. In still another aspect, the ribavirin is administered at between 1000 mg to 1200 mg per day and the pegylated interferon alpha-2a is administered in an amount of 180 micrograms once per week.

In one aspect, the present invention further provides a method of treating of a Hepatitis C virus genotype 1 infected relapser or non-responder patient with alisporivir in combination with standard of care, preferably with interferon and ribavirin, the method comprising administering alisporivir during an initial phase in an amount of about 600 mg, twice per day; followed by administering alisporivir during a second phase in an amount of about 600 to about 800 mg once per day for up to 23, 47 or 71 weeks. In still other aspects, the initial phase is a period of at least 3 days, preferably of 5 days, most preferred of 7 days.

In one aspect, the present invention further provides use of alisporivir in the manufacture of a medicament for treatment of a Hepatitis C virus genotype 1 infected relapser or non-responder patient wherein alisporivir is administered during an initial phase in an amount of about 600 mg, twice per day; followed by administering alisporivir during a second phase in an amount of about 600 to about 800 mg once per day for up 23, 47 or 71 weeks and wherein alisporivir is administered in combination with interferon and ribavirin throughout the initial and second phases. In still other aspects, the initial phase a period of at least 3 days, preferably of 5 days, most preferred of 7 days.

In one aspect, the present invention further provides use of alisporivir in the preparation of a pharmaceutical composition for treatment of a Hepatitis C virus genotype 1 infected relapser or non-responder patient characterized in that alisporivir is administered during an initial phase in an amount of about 600 mg, twice per day; followed by administering alisporivir during a second phase in an amount of about 600 to about 800 mg once per day for up to 23, 47 or 71 weeks and wherein alisporivir is administered in combination with interferon and ribavirin throughout the initial and second phases. In still another aspects, the initial phase is a period of at least 3 days, preferably of 5 days, most preferred of 7 days.

In one aspect, the present invention further provides a combination of alisporivir with standard of care, preferably with interferon and ribavirin for use in treatment of a Hepatitis C virus genotype 1 infected relapser or non-responder patient, wherein alisporivir is administered during an initial phase in an amount of about 600 mg, twice per day for 7 days; followed by administering alisporivir during a second phase in an amount of about 600 to about 800 mg once per day for up to 23, 47 or 71 weeks.

In one aspect, the present invention further provides a therapeutic regimen comprising administering alisporivir during an initial phase in an amount of about 600 mg, twice per day for one week; followed by administering alisporivir during a second phase in an amount of about 600 to about 800 mg once per day for up to 23, 47 or 71 weeks and wherein alisporivir is administered in combination with interferon and ribavirin throughout the initial and second phases.

In one aspect, the present invention further provides pharmaceutical compositions comprising alisporivir for uses as defined above. In still other aspects, the present invention provides a package comprising the pharmaceutical composition comprising alisporivir for uses as defined above in combination with instructions to administer said composition.

In exemplary embodiments, alisporivir is administered at a dosage of from about 600 to about 1000 mg twice daily for 7 days followed by administering alisporivir at a dosage of from about 600 to about 1000 mg once per day for up to 23, 47 or 71 weeks.

In exemplary embodiments, the treatment of the present invention involves administration of interferon alpha that is a pegylated interferon alpha-2a and the amount of pegylated interferon alpha-2a administered is from 20 to 250 micrograms per week on a weekly, three times a week, every other day or daily basis. The current approved dose is 180 micrograms per week. In other exemplary embodiments, the interferon alpha is a pegylated interferon alpha-2b and the amount of pegylated interferon alpha-2b is from 0.5 to 2.0 micrograms/kilogram per week on a weekly, three times a week, every other day or daily basis. Exemplary descriptions of such treatments are described in U.S. Pat. No. 7,115,578, incorporated herein by reference in its entirety.

An exemplary Peg-IFNα2a used in the treatment protocols described herein is Pegasys®. PEGASYS® is a pegylated form of IFNα2a (peg-IFNα2a) and utilizes a 40 kDa branched PEG (polyethylene glycol) to provide sustained serum concentrations for a full week (168 hours). PEGASYS® is commercially available, presented as single use, pre-filled syringes containing 180 μg/0.5 mL peg-IFNα2a for S.C. injection. The standard package contains 1 syringe of 180 μg/0.5 mL.

In some embodiments, it may be desirable to modify the dose of Peg-IFNα2a. If dose modification is required for moderate to severe adverse reactions (clinical and/or laboratory), initial dose reduction from 180 to 135 μg is generally adequate (adjustment to the corresponding graduation mark on pre-filled syringe). However, in some cases, dose reduction to 90 μg may be needed. Following improvement, re-escalation of the dose may be considered.

In treatment described above effective dosages of the standard of care agents are administered in compositions, i.e. they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the drugs as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic or additive therapeutic effect, compared to a monotherapy applying only one of its pharmaceutically active ingredients or as compared to the current standard of care therapy. The treatment used in the methods described herein may be administered by any conventional route. One or more components may be administered parentally, e.g., in the form of injectable solutions or suspensions, or in the form of injectable deposit formulations. Preferably, alisporivir will be administered orally in the form of solutions or suspensions for drinking, tablets or capsules. Pharmaceutical compositions for oral administration comprising alisporivir typically further comprise one or more pharmaceutically acceptable carrier substances. Typically, these compositions are concentrated and need to be combined with an appropriate diluent, e.g., water, prior to administration. Pharmaceutical compositions for parenteral administration typically also include one or more excipients. Optional excipients include an isotonic agent, a buffer or other pH—controlling agent, and a preservative. These excipients may be added for maintenance of the composition and for the attainment of preferred ranges of pH (about 6.5-7.5) and osmolarity (about 300 mosm/L).

The efficacy of the therapy regimen may be monitored using standard protocols. Treatment may be followed by determinations of HCV in serum and measurement of serum ALT levels. For example, the patients may be assessed for the presence of HCV RNA in their plasma. HCV RNA (IU/mL) can be measured at regular intervals during the treatment, e.g., at Day 1 (pre-dose and 4, 8, and 12 hours post-dose) and pre-dose at Day 2, Day 3, Day 8, Day 15, Day 29, and at Week 12, Week 24, Week 36, Week 48, Week 72 (when applicable), and at follow up. In addition, the HCV strains in the patient can be sequenced and assessed for identification of mutations selecting for resistance.

The endpoint of treatment is a virological response, i.e., the absence of HCV at the end of a treatment course, several months after initiation of treatment, or several months after completion of treatment. HCV in serum may be measured at the RNA level by methods such as quantitative RT-PCR or northern blots or at the protein level by enzyme immunoassay or enhanced chemiluminescence immunoassay of viral proteins. The endpoint may also include a determination of a serum ALT level in the normal range.

Exemplary treatment regimens are given in the Examples. In one exemplary regimen a subject in need of treatment is provided with pegylated interferon alfa 2a at a dose of 180 μg subcutaneously (S.C.) once weekly for 48 weeks in combination with ribavirin administered in an oral dosage of 1000/1200 mg daily (weight based) for 48 weeks and 600 mg alisporivir orally twice daily for 7 days, followed by 600 to 800 mg alisporivir orally once daily for 47 weeks.

In another exemplary protocol a subject in need of treatment is provided with pegylated interferon alfa 2a at a dose of 180 μg subcutaneously (S.C.) once weekly for 48 weeks in combination with ribavirin administered in an oral dosage of 1000/1200 mg daily (weight based) for 48 weeks and 600 mg alisoporivir orally twice daily for 7 days, followed by 800 mg alisporivir orally once daily for 47 weeks.

After a 4 week treatment period, based on patient response, the administration of alisporivir may be continued up to 48 or 72 weeks from the start of treatment at 600 or 800 mg per day orally or preferably the dose of alisporivir is reduced to a lesser amount in a daily dose (e.g., 400 or 600 mg) or more preferably, the administration of alisporivir is discontinued. The treatment with pegylated interferon alfa 2a and ribavirin is preferably continued for up to 48 or 72 weeks from the initiation of treatment. For example between weeks 5 to 48 or 72, the patient is administered 180 μg pegylated interferon alfa 2a S.C. orally once weekly and ribavirin administered in an oral dosage of 1000/1200 mg daily (weight based).

The following Examples illustrate the invention described hereinbefore.

### EXAMPLES

1. Compounds

Peg-IFNα2a is a pegylated form of interferon alfa 2a and utilizes 40 kDa branched PEG (polyethylene glycol) to provide sustained serum concentrations for a full week (168 hours). PEGASYS® is commercially available from Roche.

Ribavirin is a synthetic nucleoside analogue and is also commercially available, e.g., as COPEGUS® from Roche.

2. Clinical Study and Results

This is an international, multicentre, randomized, double-blind, placebo-controlled, 4-arm, parallel-group phase II study comparing therapy with three doses of DEB025 (600 mg QD, 800 mg QD and 400 mg BID) plus SOC (peg-IFNα2a once weekly plus RBV BID) versus triple therapy with placebo matching DEB025 plus SOC in chronic HCV GT1 patients who were non-responders to prior SOC treatment, or who have relapsed after SOC treatment.

Approximately 344 patients will be randomized into one of 4 treatment arms (A, B, C (C1/C2) and D) in a 1:1:1:1 ratio. C1 and C2 patients will be randomized in a 1:1 ratio within arm C.

The randomization will be stratified by response status to previous treatment (non-responders/relapsers), BMI (<25 kg/m2 or>25 kg/m2) and IL28B polymorphism (CC or CT/TT) at screening.

The ratio non-responders and relapsers should be kept at 50% (172 patients) of the study population.An international, multicentre, randomized, double-blind, placebo-controlled, 4-arm, parallel-group phase II study comparing triple therapy with three doses of alisporivir (400 mg, 600 mg or 800 mg) + standard of care (SOC) versus triple therapy with placebo matching alisporivir plus SOC in 258 chronic HCV GT1 patients who were non-responders to prior SOC treatment, or relapsers after SOC treatment is provided.

Patients are randomized into one of 4 treatment arms as described below in a 1:1:1:1 ratio. The randomization will be stratified by response status to previous treatment (non-responders/relapsers), BMI (<25 kg/m2 or ≧25 kg/m2) and IL28B polymorphism (CC or CT/TT) at screening. The ratio non-responders and relapsers should be kept at 50% (172 patients) of the study population.

Treatment A
Alisporivir/Placebo 3 capsules of 200 mg (600 mg) alisporivir 2×/day (BID) orally for 1 week (loading dose) followed by 3 capsules of 200 mg (600 mg) alisporivir once per day (QD) plus 1 capsule of placebo QD for 47 weeks. Only in week 17 (dummy loading) patients receive 3 capsules of 200 mg (600 mg) alisporivir in the morning and 3 capsules of placebo in the evening.
Peg-IFNα2a 180 μg subcutaneously (s.c.) once weekly for 48 weeks
Ribavirin 1000 mg/day (<75 kg) or 1200 mg/day (≧75 kg) orally in two divided doses for 48 weeks

Treatment B
alisporivir/Placebo 3 capsules of 200 mg (600 mg) alisporivir 2×/day orally for 1 week (loading dose) followed by 4 capsules alisporivir (800 mg) QD for 47 weeks. Only in week 17 (dummy loading) patients receive 3 capsules of 200 mg (600 mg) alisporivir in the morning and 1 capsule of 200 mg alisporivir plus 2 capsules of placebo in the evening.
Peg-IFNα2a 180 μg subcutaneously (s.c.) once weekly for 48 weeks
Ribavirin 1000 mg/day (<75 kg) or 1200 mg/day (≧75 kg) orally in two divided doses for 48 weeks

Treatment C1(will change to active treatment if cEVR not reached=arm C1A)
Placebo 3 capsules of placebo BID orally for 1 week (loading dose) followed by 4 capsules placebo QD for 47 weeks. Only in week 17 (dummy loading) patients receive 3 capsules of placebo in the morning and 3 capsules of placebo in the evening
Peg-IFNα2a 180 μg s.c. once weekly for 48 weeks
Ribavirin 1000 mg/day (<75 kg) or 1200 mg/day (≧75 kg) orally in two divided doses for 48 weeks

Treatment C_{1A}
Placebo alisporivir 3 capsules of placebo BID orally for 1 week (loading dose) followed by 4 capsules placebo QD for 16 weeks. After week 16 switch to:
   3 capsules of 200 mg (600 mg) alisporivir 2×/day orally for 1 week (loading dose) followed by 3 capsules of 200 mg (600 mg) alisporivir QD plus 1 capsule placebo QD for 47 weeks
Peg-IFNα2a 180 μg subcutaneously (s.c.) once weekly for 16 weeks plus 48 weeks
Ribavirin 1000 mg/day (<75 kg) or 1200 mg/day (≧75 kg) orally in two divided doses for 16 weeks plus 48 weeks

Treatment D
Placebo alisporivir 1 capsule of placebo QD (in the morning) orally for 48 weeks 2 capsules of 200 mg (400 mg) alisporivir 2×/day orally for 48 weeks
Peg-IFNα2a 180 μg s.c. once weekly for 48 weeks
Ribavirin 1000 mg/day (<75 kg) or 1200 mg/day (≧75 kg) orally in two divided doses for 48 weeks

Treatment C2 (will change to active treatment if cEVR not reached=arm C_{2A})
DEB025 placebo 3 capsules of placebo orally in the morning and 2 capsules of placebo in the evening for 48 weeks.
Peg-IFNα2a 180 μg s.c. once weekly for 48 weeks
Ribavirin 1000 mg/day (<75 kg) or 1200 mg/day (≧75 kg) orally in two divided doses for 48 weeks

Treatment C_{2A}
Placebo 3 capsules of placebo orally as morning dose and 2 capsules of placebo as evening dose for 16 weeks. After week 16 switch to:
alisporivir 2 capsules of 200 mg (400 mg) alisporivir BID orally plus 1 capsule of placebo in the morning for 48 weeks
Peg-IFNα2a 180 μg subcutaneously (s.c.) once weekly for 16 weeks plus 48 weeks
Ribavirin 1000 mg/day (<75 kg) or 1200 mg/day (≧75 kg) orally in two divided doses for 16 weeks plus 48 weeks

Primary efficacy endpoint: proportion of patients achieving cEVR (complete early virologic response) after 12 week triple therapy with DEB025 600 mg QD plus SOC versus triple therapy with placebo matching DEB025 plus SOC.

461 patients have been randomized into one of 4 treatment arms (A, B, C (C1/C2) and D) in a 1:1:1:1 ratio. C1 and C2 patients have been randomized in a 1:1 ratio within arm C.

Arm A corresponds to treatment A above (DEB 600 QD);

Arm B corresponds to treatment B above (DEB 800 QD);

Arm C corresponds to treatment C above, is a control arm with peg-IFNα2a/RBV plus placebo (Placebo+PR);

Arm D corresponds to treatment D above (DEB 400 BID).

The randomization was stratified by response status to previous treatment (non-responders/relapsers), BMI (<25 kg/m2 or ≧25 kg/m2) and IL28B polymorphism (CC or CT/TT) at screening.

In the full randomized study population 57% of the patients are non-responder and 43% relapser.

Out of the total 461 patients randomized in the study, we report the results of the first 337 randomized patients with up to week 12 on-treatment data.

Of all the 337 patients in randomized set, 38.9% was relapser while 55.2% was non-responder.

At Week 4, all the DEB025 treatment arms had >20% patients achieved RVR while 5.3% of Placebo patients achieved RVR. At Week 12, all the DEB025 treatment arms had ≧79% patients achieved EVR while 62.7% of Placebo patients achieved RVR. Among the 4 treatment arms, DEB 400 BID arm had the highest proportion in achieving RVR (37.2%), cEVR (70.5%) and EVR (80.8%) while the Placebo arm had the lowest proportion in achieving RVR, cEVR and EVR.

TABLE 1
Patients who achieved virologic response
by limit of quantitation (LOQ)

| | Arm A | Arm B | Arm C | Arm D |
|---|---|---|---|---|
| | DEB 600 QD | DEB 800 QD | Placebo | DEB 400 BID |
| | N = 81 | N = 84 | N = 75 | N = 78 |
| Variable | n (%) | n (%) | n (%) | n (%) |
| RVR | 19 (23.5) | 18 (21.4) | 4 (5.3) | 29 (37.2) |
| pEVR | 28 (34.6) | 24 (28.6) | 27 (36.0) | 8 (10.3) |
| EVR | 64 (79.0) | 72 (85.7) | 47 (62.7) | 63 (80.8) |
| cEVR | 36 (44.4) | 48 (57.1) | 20 (26.7) | 55 (70.5) |
| (primary | | | | |
| endpoint) | | | | |

RVR (rapid virologic response) is defined as HCV RNA < LOQ (25 IU/mL) after 4 weeks of treatment.
pEVR (partial early virologic response) is defined as HCV RNA decrease >= 2 log10 from baseline and > LOQ (25 IU/mL) after 12 weeks of treatment.
EVR is defined as HCV RNA decrease >= 2 log10 from baseline or < LOQ (25 IU/mL) after 12 weeks of treatment.
cEVR (complete early virologic response) is the primary endpoint of the study, defined as HCV RNA < LOQ (25 IU/mL) after 12 weeks of treatment.

1. Alisporivir for use in treatment of a Hepatitis C virus genotype 1 infected patient in combination with standard of care characterized in that
(i) the patient is a relapser or a non-responder patient, and
(ii) alisporivir is administered during an initial phase in an amount of about 600 mg, twice per day; followed by administering alisporivir during a second phase in an amount of about 600 to about 1000 mg once per day.

2. Alisporivir for use according to claim 1 characterized in that alisporivir is administered during an initial phase in an amount of about 600 mg, twice per day for 7 days; followed by administering alisporivir during a second phase in an amount of about 600 or about 1000 mg or 800 mg once per day for up to 23, 47 or 71 weeks.

3. Alisporivir for use according to claim 2 characterized in that alisporivir is administered during an initial phase in an amount of about 600 mg, twice per day for 7 days; followed by administering alisporivir during a second phase in an amount of 600 mg once per day for up to 47 weeks.

4. Alisporivir for use according to claim 1, wherein the standard of care is a combination of interferon with ribavirin.

5. Alisporivir for use according to claim 4, wherein said interferon is pegylated interferon alpha-2a, and is administered in an amount of 180 micrograms once per week.

6. Alisporivir for use according to claim 4, wherein said ribavirin is administered at between 1000 mg to 1200 mg per day.

7. A method of treating of a Hepatitis C virus genotype 1 infected relapser or non-responder patient with alisporivir in combination with standard of care, the method comprising administering alisporivir during an initial phase in an amount of about 600 mg, twice per day for 7 days; followed by administering alisporivir during a second phase in an amount of about 600 to about 1000 mg once per day for up to 23, 47 or 71 weeks.

8. Use of alisporivir in the manufacture of a medicament for treatment of a Hepatitis C virus genotype 1 infected patient characterized in that
(i) the patient is a relapser or a non-responder patient and
(ii) alisporivir is administered during an initial phase in an amount of about 600 mg, twice per day for 7 days; followed by administering alisporivir during a second phase in an amount of about 600 to about 800 mg once per day for up to 23, 47 or 71 weeks
and wherein alisporivir is administered in combination with standard of care throughout the initial and second phases.

9. A combination of alisporivir, and standard of care for use in treatment of a Hepatitis C virus genotype 1 infected relapser or non-responder patient characterized in that alisporivir is administered during an initial phase in an amount of about 600 mg, twice per day for 7 days; followed by administering alisporivir during a second phase in an amount of about 600 to about 800 mg once per day for up to 23, 47 or 71 weeks.

10. A therapeutic regimen comprising administering alisporivir during an initial phase in an amount of about 600 mg, twice per day for 7 days; followed by administering alisporivir during a second phase in an amount of about 600 to about 800 mg once per day for up to 23, 47 or 71 weeks and wherein alisporivir is administered in combination with standard of care throughout the initial and second phases.

11. A pharmaceutical composition comprising alisporivir for use according to claim 1.

12. A package comprising the pharmaceutical composition according to claim 11 in combination with instructions to administer said composition.
